(19) United States
(12) Patent Application Publication
(10) Pub. No.: US 2011/0243821 A1
(43) Pub. Date: Oct. 6, 2011

(54) CARBON DIOXIDE REDUCTION

(75) Inventors: Yugen Zhang, Nanos (SG); Jackie Y Ying, Nanos (SG); Siti Nurhanna Binti Riduan, Nanos (SG)

(73) Assignee: Agency for Science, Technology and Research, Connexis (SG)

(21) Appl. No.: 13/061,938

(22) PCT Filed: Sep. 3, 2009
(86) PCT No.: PCT/SG2009/000313

§ 371 Date: Jun. 14, 2011

Related U.S. Application Data
(60) Provisional application No. 61/136,405, filed on Sep. 3, 2008.

(51) Int. Cl. B01D 53/62; C07F 7/08; C07C 29/10

(52) U.S. Cl. 423/228; 556/466; 568/907

(57) ABSTRACT

The invention provides a process for reducing carbon dioxide comprising the step of exposing the carbon dioxide to a silane in the presence of an N-heterocyclic carbene (NHC) or a carboxylate thereof or both, to produce a methylsilyl ether.

### TECHNICAL FIELD

The present invention relates to a process for reducing carbon dioxide.

### BACKGROUND OF THE INVENTION

Carbon dioxide is a non-toxic, non-combustible, non-flammable gas that is a stable end-product of metabolism and combustion. It is abundant in the atmosphere and is known to be a greenhouse gas (GHG) that causes global warming. A process that could reduce the carbon dioxide content in the atmosphere so as to combat global warming would be very attractive, especially if such process could also generate useful commodities or fine chemicals. Large amounts of carbon dioxide are produced by burning of fuels. The direct conversion of carbon dioxide to fuels would realize a carbon-neutral source of energy which would not compete with food agriculture. However, carbon dioxide is a very stable molecule, and has found limited usage as a feedstock so far.

Catalytic reduction of carbon dioxide with hydrosilanes proceeds exothermically and provides a possible utilization of carbon dioxide in industrial chemical processes. The development of highly active and robust catalysts for such a reaction remains a major scientific challenge. Previous reports of carbon dioxide addition to hydrosilanes included the use of active transition metal complexes as catalysts. Ruthenium and iridium complexes were first reported in early 1980s as catalysts for the hydrosilylation of carbon dioxide. More recently, hydrosilylation of carbon dioxide catalyzed by ruthenium-acetonitrile complexes was reported by Pitter and co-workers, yielding formoxysilanes (Deglmann, P.; Ember, E.; Hofman, P.; Pitter, S.; Walter, O. Chem. Eur. J. 2007, 13, 2864; Jansen, A.; Gorls, H.; Pitter, S. Organometallics 2000, 19, 135). Matsuo and Kawaguchi reported the homogeneous reduction of carbon dioxide with hydrosilanes catalyzed by zirconium-borane complexes, yielding methane (Matsuo, T.; Kawaguchi, H. J. Am. Chem. Soc. 2006, 128, 12362). In these different systems, practical applications were limited by the air and moisture sensitivity and the low activities of the organometallic catalysts involved.

There is therefore a need for an improved method for reducing, or fixing, carbon dioxide, such method preferably producing useful products.

### OBJECT OF THE INVENTION

It is the object of the present invention to substantially overcome or at least ameliorate one or more of the above limitations.

### SUMMARY OF THE INVENTION

In a first aspect of the invention there is provided a process for reducing carbon dioxide comprising the step of exposing the carbon dioxide to a silane in the presence of an N-heterocyclic carbene (NHC) or a carboxylate thereof or both, to produce a methylsilyl ether.

The following options may be used in combination with the first aspect, either individually or in any suitable combination.

The process may comprise hydrolysing the methylsilyl ether to generate methanol. The step of hydrolysing may be conducted under basic conditions.

The NHC or carboxylate thereof may be catalytic. It may have been used in a previous reaction.

The NHC may be metal free. It may be an N,N′-disubstituted imidazolidin-2-ylidene or an N,N′-disubstituted imidazol-2-ylidine. It may be a dimeric NHC. It may be an oligomeric NHC. It may be a polymeric NHC (polyNHC). It may be a metal free polyNHC.

The carbon dioxide may be exposed to the silane in the presence of the carboxylate of the NHC. In this event, the process may comprise the step of reacting the NHC with carbon dioxide to generate the carboxylate of the NHC.

The process may comprise the step of generating the NHC from a corresponding N-heterocyclic salt by reacting said salt with a base. The NHC may be generated from the salt in situ. The base may be a non-nucleophilic base. It may for example be hydride (e.g. sodium or potassium hydride) or t-butoxide (e.g. sodium or potassium t-butoxide).

The silane may be used in molar excess over the carbon dioxide. Alternatively the carbon dioxide may be used in molar excess over the silane. The silane and the carbon dioxide may be used in approximately equimolar amounts.

The silane may be a diorganosilane. In this case, the process may comprise converting the diorganosilane to an oligodiorganosiloxane or a polydiorganosiloxane or a cyclooligodiorganosiloxane or a mixture of any two or all of these.

The carbon dioxide may be present in a mixture of gases. The mixture of gases may comprise oxygen or it may contain substantially no oxygen.

The NHC or carboxylate thereof may be polymeric. The polymeric NHC from a previous reaction may be treated with a strong base so as to regenerate said NHC prior to exposing said NHC to the carbon dioxide.

In an embodiment there is provided a process for reducing carbon dioxide comprising the step of exposing the carbon dioxide to a silane in the presence of an N,N′-disubstituted imidazolidin-2-ylidene or an N,N′-disubstituted imidazol-2-ylidine or a carboxylate of either of these, to produce a methylsilyl ether.

In another embodiment there is provided a process for reducing carbon dioxide comprising the step of exposing the carbon dioxide to a silane in the presence of an N,N′-disubstituted imidazol-2-ylidine carboxylate, to produce a methylsilyl ether.

In another embodiment there is provided a process for reducing carbon dioxide comprising:
reacting an N,N′-disubstituted imidazol-2-ylidine with carbon dioxide to generate a corresponding N,N′-disubstituted imidazol-2-ylidine carboxylate, and
exposing carbon dioxide to a silane in the presence of the N,N′-disubstituted imidazol-2-ylidine carboxylate, to produce a methylsilyl ether.

In another embodiment there is provided a process for reducing carbon dioxide comprising:
reacting an N,N′-disubstituted imidazol-2-ylidinium salt with a base to generate an N,N′-disubstituted imidazol-2-ylidine,
reacting the MN'-disubstituted imidazol-2-ylidine with carbon dioxide to generate a corresponding N,N′-disubstituted imidazol-2-ylidine carboxylate, and
exposing carbon dioxide to a silane in the presence of the N,N′-disubstituted imidazol-2-ylidine carboxylate, to produce a methylsilyl ether.

In another embodiment there is provided a process for reducing carbon dioxide comprising:
exposing the carbon dioxide to a silane in the presence of an N,N′-disubstituted imidazol-2-ylidine carboxylate, to produce a methylsilyl ether, and
hydrolysing the methylsilyl ether to form methanol.

In another embodiment there is provided a process for reducing carbon dioxide comprising:
reacting an N,N′-disubstituted imidazol-2-ylidinium salt with a base to generate an N,N′-disubstituted imidazol-2-ylidine,
reacting the N,N′-disubstituted imidazol-2-ylidine with carbon dioxide to generate a corresponding N,N′-disubstituted imidazol-2-ylidine carboxylate,
exposing carbon dioxide to a silane in the presence of the N,N′-disubstituted imidazol-2-ylidine carboxylate, to produce a methylsilyl ether, and
hydrolysing the methylsilyl ether to form methanol.

In another embodiment there is provided a process for reducing carbon dioxide comprising:
reacting an N,N′-disubstituted imidazol-2-ylidinium salt with a base to generate an INN′-disubstituted imidazol-2-ylidine,
reacting the N,N′-disubstituted imidazol-2-ylidine with carbon dioxide to generate an corresponding N,N′-disubstituted imidazol-2-ylidine carboxylate,
exposing carbon dioxide to a diorganosilane in the presence of the N,N′-disubstituted imidazol-2-ylidine carboxylate, to produce a methylsilyl ether, and
hydrolysing the methylsilyl ether to form methanol.

In a second aspect of the invention there is provided a method of at least partially removing carbon dioxide from a gas comprising carbon dioxide, said method comprising exposing a silane to said gas in the presence of an N-heterocyclic carbene (NHC) or a carboxylate thereof or both.

The method may comprise the step of removing water vapour from the gas prior to the step of exposing.

The gas may be air. It may be waste gas or exhaust gas from an industrial process. It may be waste gas or exhaust gas from a combustion process.

### BRIEF DESCRIPTION OF THE DRAWINGS

Preferred embodiments of the present invention will now be described, by way of an example only, with reference to the accompanying drawings wherein:

FIG. 1 shows ¹³C NMR spectra of NMR tube reactions of ¹³CO₂, diphenylsilane and Imes-CO₂ catalyst (1,3-bis-(2,4,6-trimethylphenyl)imidazolium carboxylate; 5 mol %) in DMF-d₇. Spectra A, B and D are proton decoupling spectra and spectrum C shows spectrum B in the absence of proton decoupling. Spectra A and B show the conversion of ¹³CO₂ (*) to ¹³CH₂(OSiR₃)₂ (▾) and ¹³CH₃O—SiR₃ (#). Spectrum D shows the spectrum after additional silane was added to the mixture of spectrum B, indicating that all ¹³CO₂ was converted to ¹³CH₃O—SiR₃.

FIG. 2 shows a proposed catalytic cycle and reaction pathway for the reaction described herein.

FIG. 3 shows a proton NMR spectrum of an NMR tube reaction with ¹³CO₂, diphenylsilane and Imes-CO₂ catalyst (5 mol %) in DMF-d7 after 90 min.

FIG. 4 shows a proton NMR spectrum of an NMR tube reaction with ¹³CO₂, diphenylsilane and Imes-CO₂ catalyst (5 mol %) in DMF-d7 after 24 h.

FIG. 5 shows a GC-MS spectrum after 18 h of reaction. Reaction conditions: CO₂ balloon, 1 mmol of Ph₂SiH₂, Imes-CO₂ catalyst (10 mol %), 2 mmol of PhOH, and 2 ml of DMF.

FIG. 6 shows intermediates observed in GC-MS spectrum after 1 h of reaction. Reaction conditions: CO₂ balloon, 1 mmol of Ph₂SiH₂, Imes-CO₂ catalyst (10 mol %), and 2 ml of THF.

FIG. 7 shows a GC-MS spectrum after 18 h of reaction. Reaction conditions: CO₂ balloon, 1 mmol of Ph₂SiH₂, Imes-CO₂ catalyst (10 mol %), and 2 ml of DMF.

FIG. 8 shows a GC-MS spectrum after 18 h of reaction. Reaction conditions: CO₂/O₂ (volume ratio=1:1) balloon, 1 mmol of Ph₂SiH₂, Imes-CO₂ catalyst (10 mol %), and 2 ml of DMF. All Ph₂SiH₂ was consumed. The peak at 6.8 min is associated with the external standard.

FIG. 9 is a graph showing reaction time required for the full consumption of Ph₂SiH₂ in the specified run of Example 3. Reaction conditions: 1 mmol of diphenylsilane, 10 mol % of catalyst loading, CO₂ balloon, 2 ml of solvent, room temperature. Ph₂SiH₂ was not fully consumed after an overnight reaction in run #5.

### DETAILED DESCRIPTION OF THE PREFERRED EMBODIMENTS

The present invention provides a new technique for converting carbon dioxide to methanol with silane as the hydrogen source. It represents the first carbon dioxide reduction reaction catalyzed by N-heterocyclic carbene (NHC) organocatalysts. It demonstrates a chemical carbon dioxide fixation protocol which provides the possibility of direct conversion of carbon dioxide (from air) to methanol with the formation of polysiloxanes. In the present context, “reduction” of carbon dioxide (and related terms such as “reduce” and “reducing”) may refer to removal of oxygen from the carbon dioxide. It may represent a reduction of the carbon atom of the carbon dioxide. It may represent a reduction in the number of oxygen atoms directly attached to the carbon atom of the carbon dioxide. It may represent a reduction in the number of carbon-oxygen bonds to the carbon atom of the carbon dioxide (where a carbon-oxygen double bond is considered to represent two carbon-oxygen bonds).

In the past organometallic catalysts have been examined for the reduction of carbon dioxide with silanes. Compared to transition metal catalysts, the NHC catalysts of the present invention are metal-free, less expensive, and superior in efficiency. They also allow for milder and more flexible reaction conditions and are air-tolerant. They further provide highly selective production of end-products. Benefits in providing a metal-free system include cost reduction, environmental benefits, simplicity of operation and reduction in toxic wastes. The reaction described herein can be applied towards carbon dioxide fixation. It uses carbon dioxide as a chemical feedstock and can convert carbon dioxide to methanol.

The present invention provides a process for reducing carbon dioxide comprising the step of exposing the carbon dioxide to a silane in the presence of an N-heterocyclic carbene (NHC) or a carboxylate thereof or both, to produce a methylsilyl ether. In the present specification the term “silane” is used to mean a compound having at least one Si—H bond per molecule. The term “organosilane” is used to mean a silane having at least one organic group (e.g. an alkyl group or an aryl group) directly attached to the silicon atom. Organosilanes therefore have at least one Si-organic bond and at least one Si—H bond per molecule. In many cases organosilanes will have a single silicon atom per molecule, so that the at least one organic group and the at least one Si—H bond are attached to the same silicon atom. In the event that an organosilane has more than one silicon atom, the Si—H and the organic group may be attached to the same silicon atom or to different silicon atoms. Oligodiorganosiloxanes, polydiorganosiloxanes and cyclooligodiorganosiloxanes are oligomers (optionally cyclic oligomers) and polymers with repeat units of structure —O—Si(R₂)—. In these structures the R groups on silicon are commonly the same but may be different, and may be alkyl or aryl, optionally substituted. These species commonly do not contain SiH groups, although in some instances they may. The term “methylsilyl ether” is used to refer to a compound comprising a CH₃—O—Si group. Methylsilyl ethers may or may not have a Si—H bond in their molecules.

The step of exposing the carbon dioxide to the silane may be conducted for about 1 to about 20 hours, or about 1 to 10, 1 to 5, 1 to 2, 2 to 20, 50 to 20, 10 to 20, 2 to 10, 5 to 10 or 5 to 15 hours, e.g. about 1, 2, 3, 4, 5, 6, 7, 8, 9, 10, 15 or 20 hours. It may be conducted for longer than this time, although the above times are typical for the time required to fully consume the silane in the event that there is a molar excess of carbon dioxide over silane. The time will depend on the nature of the NHC and on the temperature used. The temperature may be about 10 to about 50° C., or about 10 to 30, 10 to 20, 20 to 50, 30 to 50 or 20 to 30° C., e.g. about 10, 15, 20, 25, 30, 35, 40, 45 or 50° C.

The methylsilyl ether generated in the process may be hydrolysed to generate methanol. This may be conducted by addition of water or an aqueous mixture to the methylsilyl ether. It may be conducted in situ or may be conducted as a separate step. The step of hydrolysing may be conducted under basic conditions. It may be conducted by addition of a base (e.g. an aqueous base) to the reaction mixture containing the methylsilyl ether. Alternatively the methylsilyl ether may be at least partially separated from the reaction mixture, or at least partially purified, prior to the addition of the base. The base may be an inorganic base. It may be a hydroxide. It may be aqueous. It may be for example aqueous sodium hydroxide. The base may be used in molar excess over the methylsilyl ether. It may be used in at least about 1.5 fold molar excess, or at least about 1.75, 2, 2.5 or 3 molar excess (or about 1 to 3, 1 to 2, 2 to 3 or 1.5 to 2.5 fold molar excess, e.g. about 1.1, 1.2, 1.3, 1.4, 1.5, 2, 2.5 or 3 fold molar excess) over the methylsilyl ether. The hydrolysis may be conducted at room temperature or at any other suitable temperature. It may be conducted at about 10 to about 80° C., or about 10 to 50, 10 to 30, 10 to 20, 20 to 80, 50 to 80, 20 to 50, 20 to 30 or 60 to 70° C., e.g. about 10, 15, 20, 25, 30, 35, 40, 45, 50, 55, 60, 65, 70, 75 or 80° C. In the event that it is conducted above the boiling point of methanol (which at 1 atmosphere pressure is about 65° C.) the methanol may be continuously distilled from the reaction mixture as the hydrolysis proceeds. The hydrolysis may take from about 1 to about 24 hours, depending in part on the temperature used in the hydrolysis. It may take about 1 to 12, 12 to 24, 6 to 18 or 18 to 24 hours, e.g. about 1, 6, 12, 18 or 24 hours.

The NHC or carboxylate thereof may be used in catalytic amounts. It may be used in about 0.1 to about 10% molar equivalent relative to carbon dioxide, or about 0.1 to 5, 0.1 to 2, 0.1 to 1, 0.5 to 10, 1 to 10, 5 to 10, 0.5 to 5, 0.5 to 2 or 1 to 2%, e.g. about 0.1, 0.2, 0.3, 0.4, 0.5, 0.6, 0.7, 0.8, 0.9, 1, 1.5, 2, 2.5, 3, 3.5, 4, 4.5, 5, 6, 7, 8, 9 or 10% molar equivalent. It may be used in about 1 to about 25% molar equivalent relative to the silane, or about 1 to 20, 1 to 10, 1 to 5, 5 to 25, 10 to 25, 5 to 20 or 5 to 10%, e.g. about 1, 2, 3, 4, 5, 10, 15, 20 or 25% molar equivalent. The catalyst may be recycled, i.e. it may have been used in a previous reaction. In particular, an NHC may be reused in subsequent reactions. The catalyst may retain at least about 80% of its activity in a subsequent reaction, or at least about 85, 90 or 95% of its activity. The process may comprise regenerating the NHC if its activity has been diminished. The regenerating may comprise exposing the NHC to a base. The base may be as described for generation of the NHC from the N-heterocyclic salt (see below). Thus it may be a non-nuclophilic base. It may be a strong base. It may be a strong non-nucleophilic base, e.g. hydride or t-butoxide.

The NHC may be metal free. It may be transition metal free. It may be monomeric. It may be dimeric. It may be oligomeric. It may be polymeric. It may be soluble in the reaction mixture or may be insoluble therein, in which case it may be used as a heterogeneous catalyst. In particular, polymeric NHCs or their carboxylates may be used as heterogeneous catalysts. The NHC may be a stable NHC. The NHC carboxylate may be a stable NHC carboxylate, or it may be the carboxylate of a stable NHC, or it may be both. In this context “stable” may indicate that it may be exposed to air and/or moisture without substantial (e.g. greater than about 10%, or 5, 2 or 1%) loss of activity or that it may be exposed to the above conditions without loss of substantial (e.g. greater than about 10%, or 5, 2 or 1%) chemical purity. The exposure may be at least about 5 minutes, or at least about 10 minutes or at least about 1, 2, 6 or 12 hours. It may be at a temperature of about 10 to about 30° C., e.g. about 25° C. It may indicate stability under the conditions used in the reaction. The NHC may be an N,N′-disubstituted imidazolidin-2-ylidene or an N,N″-disubstituted imidazol-2-ylidine or a dimer, oligomer or polymer of either or both of these. The substituents on the two nitrogen atoms may be the same or may be different. They may, independently, be alkyl groups, aryl groups, heteroaryl groups or some other type of group. Suitable alkyl groups include C1-C6 straight chain alkyl groups (e.g. methyl, ethyl, propyl, butyl), C3 to C6 branched chain groups (e.g. isopropyl, t-butyl, s-butyl, neopentyl) and C3 to C6 cycloalkyl groups (e.g. cyclopentyl or cyclohexyl). Suitable aryl groups include phenyl, 2,4,6-trimethylphenyl and 2,6-diisopropylphenyl. Suitable heteroaryl groups include pyridyl, thiophenyl, pyrrolyl, furyl etc. Any of the above-mentioned groups may optionally be substituted. Thus for example the substituent may be a benzyl group (i.e. a methyl group substituted with a phenyl group). The NHC may be a sterically hindered NHC. The carbene centre (e.g. C2 of an N,N′-disubstituted imidazolidin-2-ylidene or an N,N′-disubstituted imidazol-2-ylidine) may be sterically crowded. In some cases dimeric NHC's may be used. For example two imidazolylidene groups may be linked for example by a pyridine-2,6-dimethylyl linker. The remaining nitrogen atom on each imidazolylidene may be substituted as described above.

Polymeric NHCs have been described in WO2008/039154, the contents of which are incorporated herein by cross-reference. The polymeric NHC may comprise heterocyclic groups, and a monomer unit of the polymeric carbene may comprise two of the heterocyclic groups joined by a linker group. For example a suitable polymeric NHC may have structure I.

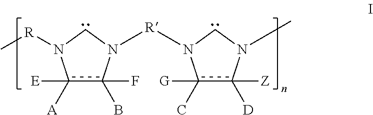

In structure I, [symbol] represents either a single or a double bond, wherein, if [symbol] represents a double bond, substituents E, F, G and Z are not present. Substituents A, B, C and D, and, if present, E, F, G and Z may each, independently, be hydrogen or a substituent which is not hydrogen. They may, independently, be hydrogen, alkyl (e.g. straight chain, branched chain, cycloalkyl), aryl (e.g. phenyl, naphthyl), halide (e.g. bromo, chloro), heteroaryl (e.g. pyridyl, pyrrolyl, furanyl, furanylmethyl, thiofuranyl, imidazolyl), alkenyl (e.g. ethenyl, 1-, or 2-propenyl), alkynyl (e.g. ethynyl, 1- or 3-propynyl, 1-, 3- or 4-but-1-ynyl, 1- or 4-but-2-ynyl etc.) or some other substituent. A, B, C and D and, if present, E, F, G and Z, maybe all the same, or some or all may be different. The alkyl group may have between about 1 and about 20 carbon atoms (provided that cyclic or branched alkyl groups have at least 3 carbon atoms), or between about 1 and 12, 1 and 10, 1 and 6, 1 and 3, 3 and 20, 6 and 20, 12 and 20, 3 and 12 or 3 and 6, e.g. 1, 2, 3, 4, 5, 6, 7, 8, 9, 10, 11, 12, 14, 16, 18 or 20 carbon atoms, and may for example be methyl, ethyl, 1- or 2-propyl, isopropyl, 1- or 2-butyl, isobutyl, tert-butyl, cyclopentyl, cyclopentylmethyl, cyclohexyl, cyclohexylmethyl, methylcyclohexyl etc. The substituents may be optionally substituted (e.g. by an alkyl group, an aryl group, a halide or some other substituent) or may comprise a heteroatom such as O, S, N (e.g. the substituent may be methoxymethyl, methoxyethyl, ethoxymethyl, polyoxyethyl, thiomethoxymethyl, methylaminomethyl, dimethylaminomethyl etc.). Substituents A, B, C and D, and, if present, E, F, G and Z may each, independently, be chiral or achiral. R and R′ in structure I are linker groups. R and R′ may each independently, be a rigid linker group or may be a non-rigid or semi-rigid linker group. Suitable rigid linker groups include aromatic groups, heteroaromatic groups, cycloaliphatic groups, suitably rigid alkenes and suitably rigid alkynes. Suitable linker groups include optionally substituted ethenyl (e.g. ethenediyl, propen-1,2-diyl, 2-butene-2,3-diyl), ethynyl (e.g. ethynediyl, propynediyl, but-2,3-yne-1,4-diyl), aryl (1,3-phenylene, 1,4-phenylene, 1,3-naphthylene, 1,4-naphthylene, 1,5-naphthylene, 1,6-naphthylene, 1,7-naphthylene, 1,8-naphthylene), heteroaryl (e.g. 2,6-pyridinediyl, 2,6-pyrandiyl, 2,5-pyrrolediyl), or cycloalkyl linker groups (e.g. 1,3-cyclohexanediyl, 1,4-cyclohexanediyl, 1,3-cyclopentanediyl, 1,3-cyclobutanediyl) groups. Suitable non-rigid or semi-rigid linker groups include —(CH₂)ₘ—, where m is between 1 and about 10, and these may be optionally substituted and/or branched, e.g. 1,2-ethanediyl, 1,2- or 1,3-propanediyl, 1,2-, 1,3-, 1,4- or 2,3-butanediyl, 2-methyl-butane-3,4-diyl etc. The linker groups may be optionally substituted (e.g. by an alkyl group, an aryl group, a halide or some other substituent) or may comprise a heteroatom such as O, S, N (e.g. a suitable linker group may be —CH₂OCH₂—, —CH₂OCH₂CH₂—, —CH₂OCH(CH₃)—, —(CH₂OCH₂)ₚ— (p between 1 and about 100), —CH₂NHCH₂—, CH₇N(CH₃)CH₂—, —CH₂N(Ph)CH₂—, —CH₂SCH₂— etc.). A general procedure for making the polyNHCs involves treating imidazole with a strong base such as NaH and treating the resulting imidazole anion in situ with a dihalo compound (e.g. 1,4-dibromobutene, α,α′-dichloro-p-xylene, etc.) to form a bisimidazole in which the imidazole groups are joined by a linker. This compound may then be polymerised by exposure to a second dihalo compound (e.g. 1,2-dibromethane, 1,4-dibromobutylene etc.). Treatment of this polymer with a base such as sodium t-butoxide provides the polyNHC. A person skilled in the art will readily appreciate suitable variations to this method which will produce polyNHCs of various structures.

The carbon dioxide may be exposed to the silane in the presence of the carboxylate of the NHC. The carboxylate may be regarded as an adduct of the NHC with carbon dioxide. In this adduct, a carboxyl (—CO₂⁻) group is attached to C2 of the NHC (e.g. of the imidazole or imidazoline ring). In the context of this specification, standard numbering of heterocyclic rings is adhered to. Thus in an imidazole or imidazolidine ring, the two nitrogen atoms are designated N1 and N3 and the carbon atom between them is designated C2. The remaining two carbon atoms are designated C4 and C5. C4 and C5 may, independently, be unsubstituted (i.e. have only hydrogen substituents) or may be substituted. They may, independently, be substituted by alkyl, aryl or heteroaryl groups as described above. They may form part of a ring which is fused to the ring of the NHC. The fused ring may have for example 5, 7 or 7 atoms (including C4 and C5). Each of the atoms other than C4 and C5 may, independently, be C or may be a heteroatom, e.g. N, O, S. The fused ring, if present, may be alicyclic, aromatic or heteroaromatic.

If the carbon dioxide is exposed to the silane in the presence of an NHC carboxylate, the process may comprise the step of reacting the NHC with carbon dioxide to generate the NHC carboxylate. This may be conducted in a solvent. The solvent may be polar. It may be aprotic. It may be a polar aprotic solvent. It may be dried before use. It may for example be DMF, DMSO, HMPT, methylene chloride, chloroform, ethylene carbonate, propylene carbonate, THF, acetonitrile, acetone, 1,4-dioxane or some other solvent. The NHC may be in solution in the solvent, or it may be in suspension, or it may be partially in suspension and partially in solution. The reaction may be conducted over about 1 to about 24 hours, or about 1 to 12, 12 to 24, 6 to 18 or 18 to 24 hours, e.g. about 1, 2, 3, 4, 5, 6, 12, 18 or 24 hours. It may be conducted at about 10 to about 50° C., or about 10 to 30, 10 to 20, 20 to 50, 30 to 50 or 20 to 30° C., e.g. about 10, 15, 20, 25, 30, 35, 40, 45 or 50° C. It may be conducted in an atmosphere of carbon dioxide or of a gas comprising carbon dioxide. The carbon dioxide, or gas comprising carbon dioxide, may be dry. It may be dried prior to use. It may have a moisture level of less than about 1000 ppm, or less than about 500, 200, 100, 50, 20 or 10 ppm. The process may comprise drying the air to this moisture level. In some cases the process may be capable of tolerating higher levels of moisture in the gas. The partial pressure of the carbon dioxide may be about 0.1 to about 1 atmosphere, or about 0.1 to 0.5, 0.1 to 0.2, 0.2 to 1, 0.5 to 1 or 0.2 to 0.5 atmosphere, or about 0.1, 0.2, 0.3, 0.4, 0.5, 0.6, 0.7, 0.8, 0.9 or 1 atmosphere. It may be more than 1 atmosphere, or may be less than 0.1 atmosphere. The carboxylate may be generated in situ. Thus in some embodiments the NHC is converted to the corresponding carboxylate as described above and the silane added directly to the reaction mixture so as to generate the methylsilyl ether. In the present specification, the term “in situ” is used to indicate that product(s) is (are) not isolated prior to further use. Thus if the carboxylate is generated in situ, this indicates that it is generated from its precursor and then used without isolation of the carboxylate.

The carbon dioxide used in the process (either for reacting with the NHC or carboxylate, or for generating the carboxylate from the NHC, or both) may be obtained from any suitable source. It may be purchased as a pure gas or clean mixture of gases. It may be, or may be obtained from, ambient air containing low levels (commonly less than about 500 ppm, but optionally greater than this) of carbon dioxide. It may be obtained by combustion of a fuel. It may for example represent, or comprise, waste gas from an industrial process. It may comprise flue gas. In certain of the above cases, the present process may represent a method for sequestering carbon dioxide, or for at least partially scrubbing a gas containing carbon dioxide so as to reduce its carbon dioxide level. In some instances a gas mixture containing carbon dioxide may be pretreated before use in the present process in order to increase the concentration of carbon dioxide therein. This may be achieved by removing other components from the mixture, e.g. by membrane separation or other suitable method. This may serve to increase the efficiency of the process described herein.

The process may comprise the step of generating the NHC from a corresponding N-heterocyclic salt. This may comprise reacting the salt with a base. The NHC may be generated from the salt in situ. Thus in some embodiments, the salt is treated with base to form the NHC. This is then treated in situ with carbon dioxide to form the NHC carboxylate, and a silane added so as to react with additional carbon dioxide to form the methylsilyl ether. As described earlier, this may be hydrolysed in situ to form methanol. Thus the reaction may be conducted as a one pot reaction starting with the N-heterocyclic salt or from the NHC and resulting in formation of the methylsilyl ether or of methanol.

The formation of the NHC may be conducted in a solvent. The solvent may be selected from the same group as described above for formation of the NHC carboxylate. The base may be a non-nucleophilic base. It may be a strong base. It may be a strong non-nucleophilic base. It may be a sufficiently strong base to generate the NHC from the N-heterocyclic salt. It may be sodium hydride or potassium hydride or sodium t-butoxide or potassium t-butoxide or some other strong non-nucleophilic base.

The silane may be used in molar excess over the carbon dioxide or it may be less than a molar equivalent relative to the carbon dioxide. The silane may be used at a molar % relative to carbon dioxide of about 10 to about 1000%, or about 10 to 100, to 50, 10 to 20, 20 to 100, 50 to 100, 100 to 1000, 500 to 1000, 100 to 500, 100 to 200, 50 to 200, 20 to 200 or 50 to 500%, e.g. about 10, 20, 30, 40, 50, 60, 70, 80, 90, 100, 150, 200, 250, 300, 350, 400, 450, 500, 600, 700, 800, 900 or 1000%. If an excess of silane is used, all of the CO₂ may be converted to methanol. If an excess of CO₂ is used, all of the silane may be converted to methanol. If equimolar amounts of silane and CO₂ are used, both may be converted to methanol, commonly in about 95% yield. Thus in certain cases the molar % may be less than 10% or greater than 1000% (e.g. about 5, 2, 1, 0.5, 0.1, 0.1, 2000, 5000 or 10000%).

The silane may have 1, 2, 3 or 4 Si—H bonds. It may be a monoorganosilane, or a diorganosilane, or a triorganosilane, or it may be silane itself. The organic group(s) on the silicon, if present, may, independently, be alkyl, aryl or heteroaryl as defined earlier. The process may produce an oligodiorganosiloxane or a polydiorganosiloxane or a cyclooligodiorganosiloxane or a mixture of any two or all of these, or a hexaorganodisiloxane or an organosilsesquioxane or silica or some other Si—O containing species. When referring above to a molar equivalence of the silane, this may be a molar equivalence in regard to silicon atoms of the silane or of the silane as a whole or of Si—H groups in the silane. In some cases the silane may be dimeric, trimeric or oligomeric. It may be for example a disilane or a trisilane, provided that at least one of the silicon atoms, optionally all of the silicon atoms, have a Si—H bond. Thus for example the silane may be 1,1,2,2-tetraphenylsilane (Ph₂(H)Si—Si(H)Ph₂). In some cases, the silane may have groups other than alkyl, aryl and heteroaryl attached to the silicon atom.

The carbon dioxide may be used neat or as a mixture with one or more other gases. The other gas(es) may be inert towards the NHC or carboxylate thereof. The carbon dioxide may be used in a mixture in which it represents between about 1 and about 99% by volume, or about 1 to 50, 1 to 20, 1 to 10, 10 to 99, 20 to 99, 50 to 99, 90 to 99, 95 to 99, 10 to 50, 50 to 90, or 80 to 90%, e.g. about 1, 2, 3, 4, 5, 6, 7, 8, 9, 10, 20, 30, 40, 50, 60, 70, 80, 90, 91, 92, 93, 94, 95, 96, 97, 98 or 99%. The mixture may be air (in which case the level of carbon dioxide may be less than 1% by volume). The carbon dioxide or mixture of gases may be dried before use. It may be deoxygenated before use. The carbon dioxide or mixture of gases may be used as an atmosphere above the reaction mixture. It may be bubbled through the reaction mixture. It may be at least partially dissolved in the reaction mixture. The present reaction may be capable of being conducted in the presence of oxygen. This renders it far more robust than earlier systems. Thus the mixture of gases may comprise oxygen. The present reaction may be capable of being conducted in the presence of some water. Thus the carbon dioxide or mixture of gases may comprise water. The reaction described herein may be conducted as a two step process. The first step generates Si—OMe (i.e. a methylsilyl ether) and the second step is a hydrolysis to generate methanol. The first step may be to some degree sensitive to water, however the second step is run in the presence of water. If about the reaction is conducted in a continuous system, the catalyst may be fixed with all reactants in a mobile phase.

The process described herein may be conducted as a batchwise process. It may be conducted as a continuous or semicontinuous process. The latter may be suitable in cases where the catalyst is a heterogeneous catalyst for example a polymeric NHC or carboxylate thereof. Thus for example a bed of catalyst may have a solution of silane passing downwards through the bed while a stream of carbon dioxide containing gas passes upwards through the bed. By adjusting the flowrates of the solution and the gas appropriately, the carbon dioxide may be consumed continuously while continuously generating methylsilyl ether. This may optionally be hydrolysed either continuously or batchwise to generate methanol. Alternatively a stream of silane solution having dissolved carbon dioxide therein may be passed through a catalyst bed to generate the methylsilyl ether continuously.

Described herein is the first organocatalyzed hydrosilylation of carbon dioxide using a stable N-heterocyclic carbene (NHC) as catalyst. Remarkably, methanol was found to be the direct end-product from air feedstock under very mild conditions. NHCs have been well established as organocatalysts in organic synthesis. Singlet carbenes with a vacant orbital can in certain cases mimic the chemical behaviour of transition metal centers, for example in splitting dihydrogen. However NHCs can behave as nucleophiles, as they have a lone pair of electrons. It has been known that nucleophilic NHCs are able to activate carbon dioxide to form imidazolium carboxylates. However, the application of such carboxylates has been limited to their use as precursors to NHC-metal complexes and halogen-free ionic liquids. Imidazolium carboxylates have also been used in stoichiometric transcarboxylation reactions. The detachment of carbon dioxide from the imidazolium carboxylates and the closing of a catalytic cycle with NHCs have not previously been achieved. In the present work, the inventors considered that a hydrosilane may be able to act as a hydride donor in order to activated carbon dioxide, eventually resulting in reduction of carbon dioxide to methanol (see Scheme 1).

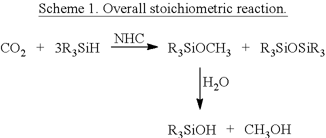

### Example 1

In a typical reaction 1,3-bis-(2,4,6-trimethylphenyl)imidazolium carboxylate (Imes-CO₂, 0.05 mmol) was dissolved in 2 mL of N,N-dimethylformamide (DMF) in a vial and carbon dioxide was introduced into the vial via a balloon. 1 mmol of diphenylsilane was introduced to the vial and the reaction mixture was stirred at room temperature. The reaction was monitored by gas chromatography-mass spectrometry (GC-MS). It was found that all diphenylsilane was fully consumed in 6 h. It was found that the expected formoxysilane product occurred as a minor product in the early stages of the reaction, and it disappeared as the reaction progressed. Further studies showed that reaction intermediate, diphenyldiformoxysilane (Ph₂Si(OCHO)₂) and diphenylformoxysilane (Ph₂SiH(OCHO)), were not stable. They underwent further reduction to bis(silyl)acetal (Si—O—CH₂—O—Si) and silylmethoxide (Si—OMe). Proton nuclear magnetic resonance (NMR) spectrum for the reaction in DMF-d₇ illustrated a major group of peaks at ˜3.5 ppm, corresponding to methoxide products. Some minor peaks at 4.5-5.0 ppm and 8.5 ppm were also identified, corresponding to silylacetal and formoxysilane intermediates. These intermediates were further confirmed by GC-MS

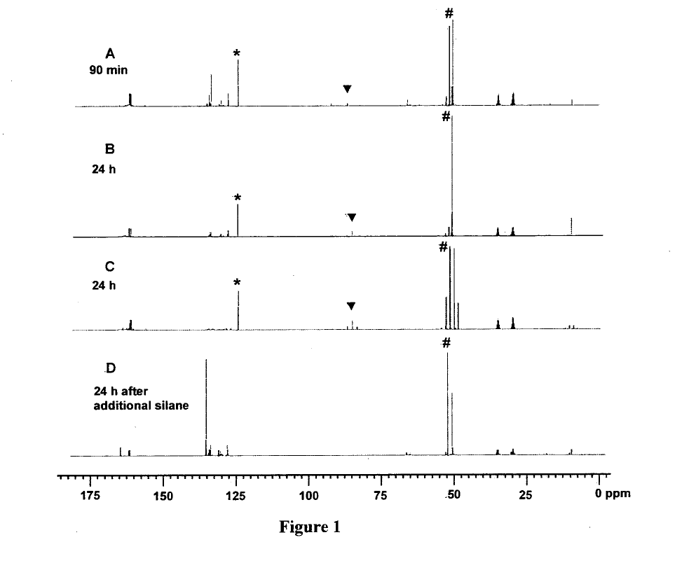

To further investigate the intervening processes of the reaction, the reaction was conducted with isotopically enriched ¹³CO₂ (99 at % ¹³C). ¹²CO₂ was introduced into an NMR tube fitted with a J. Young valve that contained 0.1 mmol of silane and 0.01 mmol of imidazolium carboxylate in DMF-d₇ solvent. The reaction was monitored with ¹³C proton decoupled NMR spectroscopy. Within 90 min, 3 groups of new peaks appeared: (i) ˜160 ppm, corresponding to the formation of formoxysilanes; (B) ˜85 ppm, indicating the formation of silylacetal intermediates, and (C) ˜50 ppm, associated with methoxide products. As the reaction progressed, the relative intensity of the peak at 85 ppm decreased, while the relative intensity of the peak at 50 ppm increased, confirming that the silylacetyl intermediates further reacted to form methoxide products (see FIG. 1). ¹³C coupled ¹H (gated decoupling) NMR experiments were also performed. The peak corresponding to 85 ppm split into a triplet and the peak at 50 ppm split into a quartet, with a coupling constant of 168.1 and 142.9 Hz, respectively. This observation clearly confirmed that CO₂ was catalytically reduced to methoxide products with hydrosilane as the hydrogen source. The reaction proceeded rapidly at room temperature. After 90 min, almost 50% of the hydrogen atoms from the hydrosilane were converted to methoxide as shown by proton NMR analysis. This conversion increased to 85% after 24 h of reaction. These results indicated that NHCs were highly efficient catalysts for this reaction, as compared to transition metal catalysts that required weeks to obtain the final reduction products. The present study also showed that an excess amount of the silane led to a much faster rate with the same final products. In this case intermediate products were not detected.

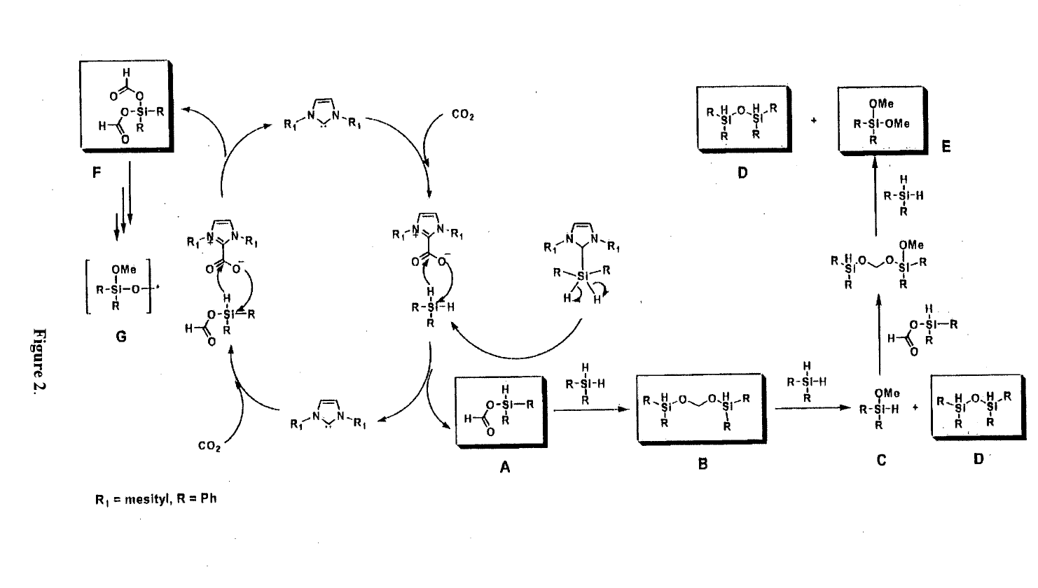

In previous work using transition metal catalysts, CO₂ reduction reaction started from metal hydride intermediate, and the reduction reaction occurred on the same metal center. The detailed mechanism for the overall catalytic system of the present invention remains unclear, but the inventors propose a possible mechanistic pathway (Scheme 2, shown in FIG. 2), without wishing to be bound to this mechanism. In this scheme, a nucleophilic carbene would activate carbon dioxide to form an imidazolium carboxylate. This adduct would then be more reactive towards silanes whereby the Si—H bond might also be activated by a free carbene. The carboxyl moiety of imidazolium carboxylate would attack the electropositive silane centre and promote hydride transfer to form a formoxysilane A and F. The formoxysilane was a key intermediate for the catalytic cycle, and would react with other free hydrosilanes in the presence of the NHC catalyst. This would result in a few other intermediates B, C and D, and the final methoxide products E and G. This catalytic cycle would continue until the supply of hydrosilane as a hydride donor has been exhausted. Intermediates A, B, D, E and F suggested in Scheme 2 have been detected by GC-MS.

Efforts to isolate the formoxysilane intermediates from the reaction were not successful due to the unstable nature and short life time of intermediates. One strategy that was assessed was to stabilize formoxysilane intermediates by introducing bulky alcohols. When the reaction mixture was spiked with phenol, a stable intermediate substituted formoxysilane (Ph₂Si(OCHO)(OC(O)OPh) was isolated as a mixture with Ph₂Si(OPh)₂ byproduct.

### Example 2

A reaction was performed with carbene catalyst generated in situ by treatment of an imidazolium salt with a strong base. The subsequent introduction of carbon dioxide to the reaction vessel gave the same activity as the imidazolium carboxylate. The reaction worked well if a non-nucleophilic base was used for the in situ generation of the carbene moiety. The counter anions from nucleophilic bases, such as potassium t-butoxide, might react with the electropositive silane to form tert-butoxide-silane adducts as undesired by-product. The reaction did not materialize when 1,8-diazabicyclo[5.4.0]undec-7-ene (DBU) was used as a base, while sodium hydride and potassium t-butoxide were found to be excellent bases for the reaction. The reaction generally worked well in polar aprotic solvents, while the use of methanol as a solvent resulted in nucleophilic methoxide addition to the hydrosilane. DMF, tetrahydrofuran (THF) and acetonitrile were found to be good solvents for the reaction, although the reaction was observed to be slower in THF and acetonitrile.

A variety of NHC ligands were examined in CO₂ reduction with diphenylsilane (Table 1). In general, all NHCs examined were effective for CO₂ reduction. The NHCs with bulky substitutions offered higher efficiencies. We have also examined CO₂ reduction by various hydrosilanes with mesitylimidazolylidene as the catalyst. The reaction was sensitive to steric hindrance around the substrate Si—H bond. Reactions with tri-substituted silanes were sluggish or inactive.

To convert carbon dioxide to methanol, the CO₂ reduction product was subjected to hydrolysis. Two equivalents of NaOH/H₂O were added to a typical CO₂ reduction mixture of diphenylsilane and mesitylimidazolylidene catalyst after a reaction period of 24 h. Methanol was produced in good yield, as characterized by GC with an external standard.

The transition metal catalysts for CO₂ reduction with silanes were usually very oxygen-sensitive, which limited their practical applications. In contrast, the present NHC catalytic system is tolerant to di-oxygen. When dry air was used as a feedstock in CO₂ reduction with diphenylsilane and mesitylimidazolylidene catalyst, the reaction proceeded smoothly to form intermediates and the methoxide product, and was complete in 7 days. Reaction with a mixed CO₂/O₂ feedstock offered the same results as that with a pure CO₂ feedstock. This demonstrated the practical applicability of the present system in the transformation of CO₂ in dry air feedstock to methanol, which would be highly attractive for industrial processes.

### Experimental

All solvents and chemicals were used as received from commercial suppliers, unless otherwise noted. Dry solvents and nitrogen glove box were used for the set up of reactions. Various imidazolium salts and silanes were purchased from Sigma-Aldrich Co. Imes-CO₂ was synthesized according to literature ((a) Holbrey, J. D.; Reichert, W. M.; Tkatchenko, I.; Bouajila, E.; Walter, O.; Tommasi, I.; Rogers, R. D. Chem. Commun. 2003, 1, 28. b) Duong, H. A.; Tekavec, T. N.; Arif, A. M.; Louie, J. Chem. Commun. 2004, 1, 112). CO₂ and O₂ were obtained from SOXAL, while ¹³C-enriched CO₂ was purchased from Sigma-Aldrich Co. GC-MS was performed on a Shimadzu GCMS QP2010 system. Gas chromatography (GC) was conducted on an Agilent GC6890N system. Centrifugation was performed on Eppendorf Centrifuge 5810R (4000 rpm, 10 min). ¹H and ¹³C NMR spectra were recorded on Bruker AV-400 (400 MHz) instrument.

### Typical Reaction Procedures

Imidazolium salt (0.25 mmol) and sodium hydride (0.25 mmol) were dissolved in 0.5 mL of solvent in a crimp top vial, and stirred for 30 min for the carbene to be generated (0.5 mmol per mL solution). The solution was then centrifuged so that the inorganic salts resulting from deprotonation would settle at the bottom. 0.2 mL of the carbene solution was transferred into a fresh vial, and 2 mL of solvent was introduced. The vial was sealed, and carbon dioxide was introduced into the vial via a balloon. The reaction was allowed to stir for 10 min, after which 1 mmol of silane was introduced. An internal standard of mesitylene was added (0.5 mmol).

Aliquots of the reaction mixture was withdrawn after specified reaction periods, and diluted with methylene chloride before the GC-MS analysis.

For conversion studies, a GC calibration curve was constructed with mesitylene and various concentrations of diphenylsilane. Aliquots were drawn from the reaction mixture at hourly intervals, and diluted with methylene chloride before the GC analysis.

For reactions with dry air, a compressed air supply was passed though a calcium sulfate drying tube before being bubbled into the reaction mixture. A sample from the reaction mixture was subjected to GC-MS analysis. An analogous reaction was also performed with air supplied from a balloon.

The reaction was tested with a variety of silanes. Reactions involving tri-substituted silanes were sluggish, with products observed only after 3 h. The reaction was also affected by the groups attached to the silane center. Triphenylsilane and diphenylmethylsilane did not react with carbon dioxide at room temperature. The order of activities for the silanes was found to be as follow: PhSiH₃>>Ph₂SiH₂>>PhSiHMe₂>Et₂SiHMe>Et₃SiH(Ph₂SiHMe and Ph₃SiH).

### Hydrolysis Reactions

To produce methanol via hydrolysis of the reaction mixture, the reaction was quenched after 18 h by adding 2 equivalents of NaOH/H₂O solution. It was stirred for another 24 h before an aliquot of isopropanol was added as an internal standard. The resulting mixture was subjected to GC analysis.

TABLE 1
Catalytic Efficiency of Various NHCs.^{a}

| Entry | Catalyst | Loading (mol %) | Time (h)^{b} |
|---|---|---|---|
| 1 | 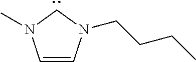 | 10 | 4 |
| 2 | 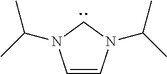 | 10 | 4 |
| 3 | 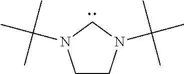 | 10 | 10 |
| 4 | 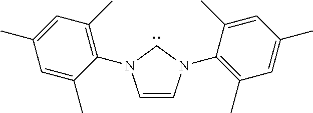 | 10 | 6 |
| 5 | 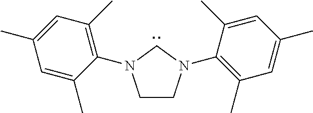 | 10 | 6 |
| 6 | 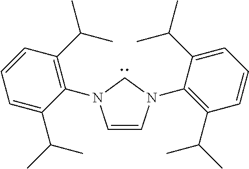 | 10 | 5 |
| 7 | 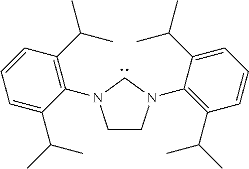 | 10 | 5 |
| 8 | 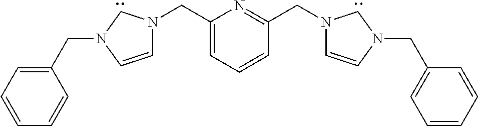 | 5 | 6 |

^{a}Reaction conditions: 1 mmol of diphenylsilane, 5-10 mol % catalyst, CO₂ balloon, 2 ml of DMF, room temperature.
^{b}Time required for the full consumption of diphenylsilane.

### NMR Tube Reaction

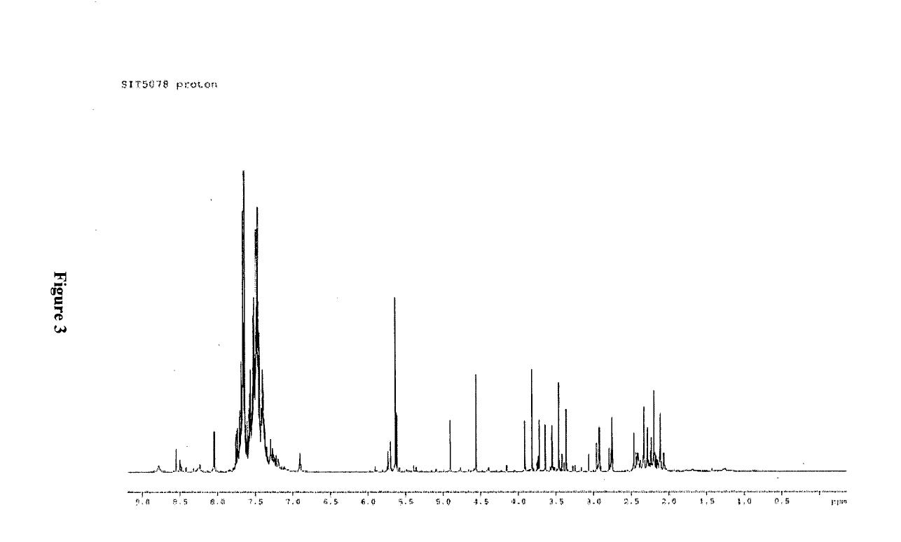

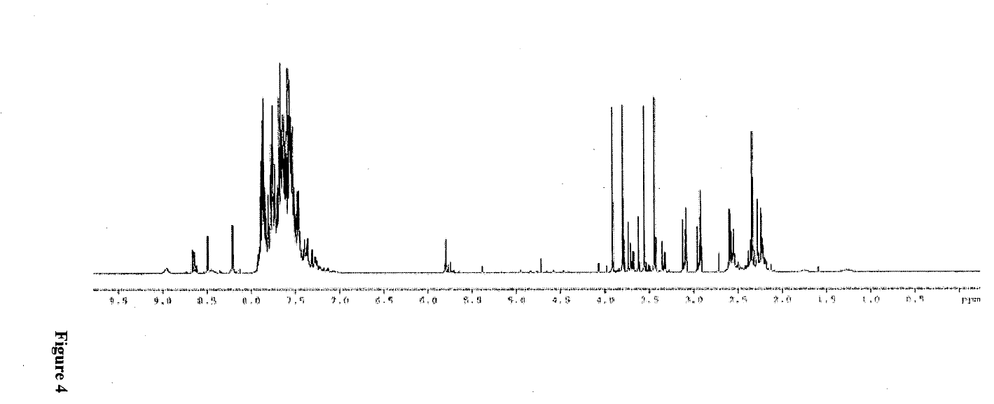

1,3-bis-(2,4,6-trimethylphenyl)imidazolium carboxylate was synthesized via the literature method, and a stock solution of Imes-CO₂ (0.05 mmol/mL) was prepared in DMF-d₇. An aliquot corresponding to 0.01 equivalent of catalyst was transferred into a NMR tube, and 0.5 mL of DMF-d₇ was added. 0.1 equivalent of silane was subsequently added, and the tube was sealed, and then evacuated and refilled with ¹³CO₂ with 2 freeze-pump-thaw cycles. The reaction was monitored via ¹³C decoupled and coupled NMR spectroscopy (see FIGS. 3 and 4).

### Isolation of Intermediates

For the isolation of intermediates, the reaction was conducted according to the procedures outlined above for a typical reaction, except that 2 equivalents of phenol were added into the mixture as a solution in DMF. The reaction was monitored via GC-MS, and the solvent was removed in vacuo. Two products were detected by GC-MS, (Ph2Si(OCHO)(OC(O)OPh), MW=364, tr=17.4 min; Ph2Si(OPh)2, MW=368, tr=21.3 min. (see FIG. 5).

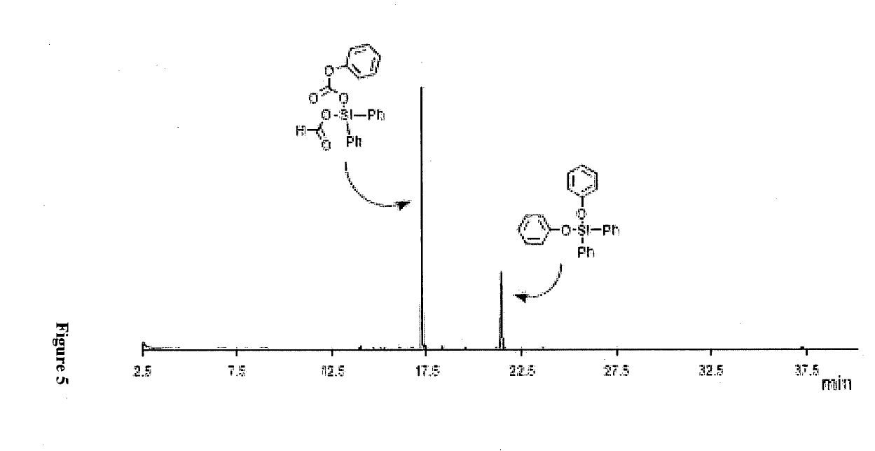

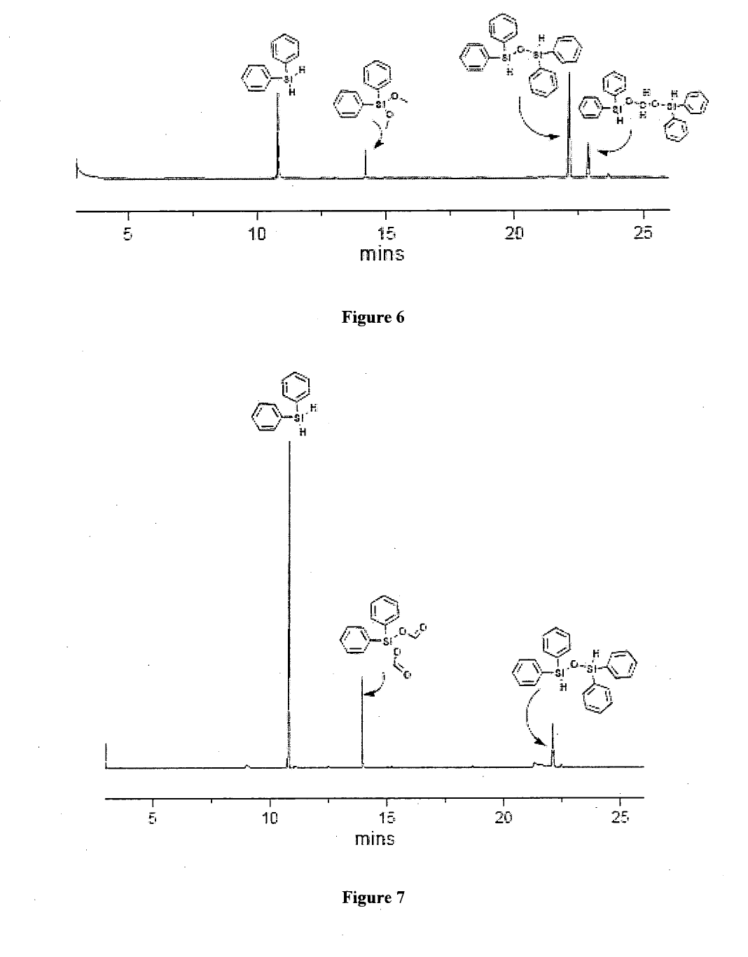

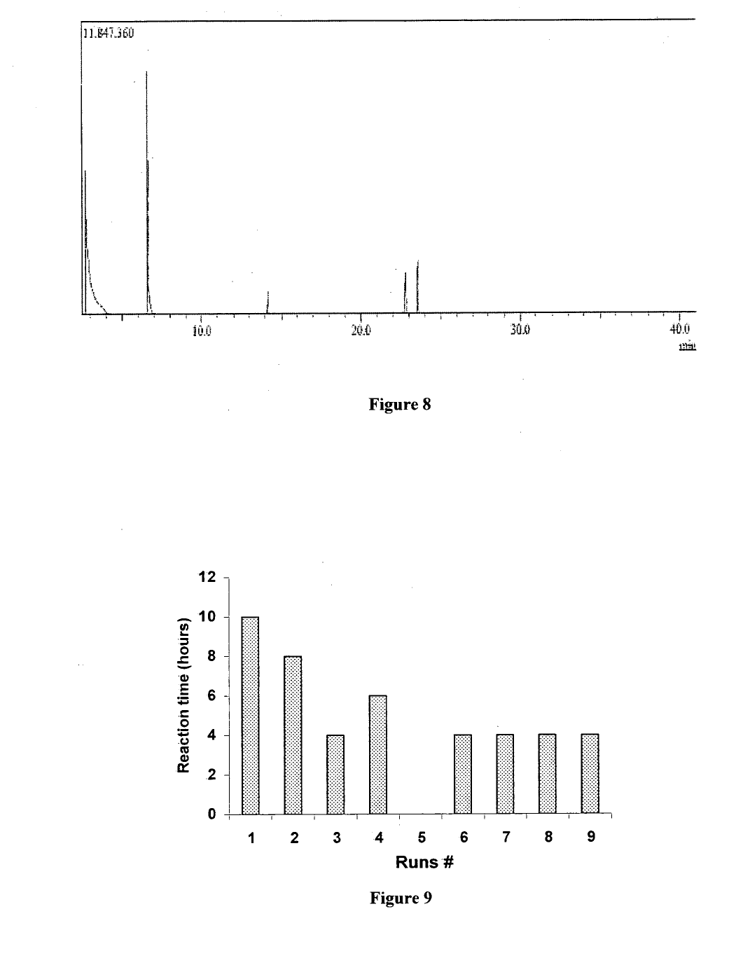

FIGS. 6 to 8 show GC-MS chromatograms of the reaction under various conditions and reaction times.

The work described herein represents the first CO₂ reduction reaction catalyzed by NHC organocatalysts. Compared to transition metal catalysts, NHCs present superior efficiency and allows for the use of milder and more flexible reaction conditions. The catalytic reduction of CO₂ with NHCs also provides for a highly selective end-product using an air-tolerant catalyst system. It offers a very promising chemical CO₂ fixation protocol, which can be applied towards the direct conversion of CO₂ in air to methanol via the formation of polysiloxanes.

### Example 3

### Conversion of Carbon Dioxide to Methanol with Silanes Over Poly-N-Heterocyclic Carbene Catalysts

The inventors have demonstrated that N-heterocyclic carbene can catalyze the conversion of carbon dioxide to methanol under ambient conditions. Herein it is shown that this conversion can be catalyzed by poly-N-heterocyclic carbene (poly-NHC) in a heterogeneous reaction system. The poly-NHC catalyst is highly efficient and can be recovered and reused multiple times. The poly-NHC was synthesized based on the method described in an earlier publication (Y. Zhang, L. Zhao, P. K. Patra, D. Hu. J. Y. Ying, Nano Today 2009, 4, 13), the contents of which are incorporated herein by cross-reference.

### Hydrosilylation of CO₂

### Preparation of Catalysts

A 1 mmol equivalent of poly-imidazolium, an equimolar amount of sodium hydride, and 10 mL of anhydrous N,N-dimethylformamide (DMF) were placed in a 20-mL crimp top vial. This vial was sealed and the suspension was stirred for 1 h before CO₂ was introduced via a balloon. The reaction mixture was allowed to stir overnight before the suspension was centrifuged and the supernatant was removed. The remaining solid was then washed with three portions of 10 mL of dichloromethane, and left under the Schlenk line to dry overnight.

The reaction used 0.1 mmol equivalent of poly-imidazolium carboxylate, and the addition of DMF (2 mL) and 1 mmol of silane in a 8-mL crimp top vial. The vial was then evacuated, and CO₂ was introduced via a balloon.

### In Situ Reactions

A 0.1 mmol equivalent of poly-imidazolium (i.e. that amount of polyimidazolium containing 1 mmol of imidazolium groups), an equimolar amount of sodium hydride, and 2 mL of anhydrous DMF were placed in an 8-mL crimp top vial. The vial was sealed and the suspension was stirred for 1 h before CO₂ was introduced via a balloon. The reaction mixture was allowed to stir for 1 h before 1 mmol of silane was added. Aliquots were withdrawn from the sample at 2-h intervals, and subjected to GC-MS analysis with mesitylene as an external standard.

### Results and Discussions

Solid poly-NHC catalyst effectively catalyzed the reaction, achieving complete consumption of Ph₂SiH₂ in 12 h. The solid catalyst was easily recycled, and the subsequent runs were much faster than the first run. The solid catalyst could be recycled for up to 5 runs. Catalyst deactivation was observed after 6 runs, whereby incomplete consumption of Ph₂SiH₂ was observed even after 12 h of reaction. Results are shown in Fig. x. However, after the regeneration of the catalyst via reaction with a strong base (NaH), the poly-NHC became highly active, and silane was fully consumed in 4 h in subsequent runs.

Nuclear magnetic resonance (NMR) and gas chromatography/mass spectrometry (GC/MS) studies showed that similar Si—OMe products were formed with the poly-NHC catalyst as with the IMes catalyst. The supernatant of the reaction mixture was collected and analyzed. Methanol was produced via hydrolysis of the reaction supernatant by adding 2 equivalents of NaOH/H₂O solution. It was stirred for another 24 h before an aliquot of isopropyl alcohol was added as an internal standard. An aliquot of 1 mL was removed from the sample, and diluted with dichloromethane before the resulting mixture was subjected to GC analysis with an Agilent HP-5 column ((5%-phenyl)-methylpolysiloxane bonded phase). 40% of methanol yield (based on silane) was achieved for each recycled run.

1. A process for reducing carbon dioxide comprising the step of exposing the carbon dioxide to a silane in the presence of an N-heterocyclic carbene (NHC) or a carboxylate thereof or both, to produce a methylsilyl ether.

2. The process of claim 1 comprising hydrolysing the methylsilyl ether to generate methanol.

3. The process of claim 2 wherein the step of hydrolysing is conducted under basic conditions.

4. The process of claim 1 wherein the NHC or carboxylate thereof is catalytic.

5. The process of claim 4 wherein the NHC or carboxylate thereof has been used in a previous reaction.

6. The process of claim 1 wherein the NHC is metal free.

7. The process of claim 1 wherein the NHC is an N,N′-disubstituted imidazolidin-2-ylidene or an N,N′-disubstituted imidazol-2-ylidine.

8. The process of claim 1 wherein the carbon dioxide is exposed to the silane in the presence of the carboxylate of the NHC and wherein the process comprises the step of reacting the NHC with carbon dioxide to generate the carboxylate of the NHC.

9. The process of claim 1 comprising the step of generating the NHC from a corresponding N-heterocyclic salt by reacting said salt with a base.

10. The process of claim 9 wherein said generating is conducted in situ.

11. The process of claim 9 wherein the base is a non-nucleophilic base.

12. The process of claim 9 wherein the base is sodium hydride or potassium t-butoxide.

13. The process of claim 1 wherein the silane is used in molar excess over the carbon dioxide.

14. The process of claim 1 wherein the silane is a diorganosilane.

15. The process of claim 14 wherein the process comprises converting the diorganosilane to an oligodiorganosiloxane or a polydiorganosiloxane or a cyclooligodiorganosiloxane or a mixture of any two or all of these.

16. The process of claim 15 wherein the carbon dioxide is present in a mixture of gases.

17. The process of claim 1 wherein the NHC or carboxylate thereof is polymeric.

18. The process of claim 17 comprising treating the polymeric NHC from a previous reaction with a strong base so as to regenerate said NHC prior to exposing said NHC to the carbon dioxide.

19. A method of at least partially removing carbon dioxide from a gas comprising carbon dioxide, said method comprising exposing a silane to said gas in the presence of an N-heterocyclic carbene (NHC) or a carboxylate thereof or both.

20. The method of claim 19 comprising the step of removing water vapour from the gas prior to the step of exposing.
